(19) United States
(12) Patent Application Publication
(10) Pub. No.: US 2003/0055293 A1
(43) Pub. Date: Mar. 20, 2003

(54) Method for epoxidizing olefins

(76) Inventors: Hanns Wurziger, Darmstadt (DE); Guido Pieper, Darmstadt (DE); Norbert Schwesinger, Eching (DE)

Correspondence Address: MILLEN, WHITE, ZELANO & BRANIGAN, P.C., 2200 CLARENDON BLVD., SUITE 1400, ARLINGTON, VA, 22201, US

(21) Appl. No.: 10/258,745

(22) PCT Filed: Apr. 5, 2001
(86) PCT No.: PCT/EP01/03875

(30) Foreign Application Priority Data
Apr. 27, 2000 (DE) 100 20 632.8

(51) Int. Cl. C07C 45/00

(52) U.S. Cl. 568/451

(57) ABSTRACT

The present invention relates to a process for the epoxidation of olefins.

The present invention relates to a process for the epoxidation of olefins.

The epoxidation of olefins is a very common process in the chemical industry and its great importance is also reflected in numerous publications on this subject.

However, epoxidations carried out on the industrial scale have safety problems and dangers associated with them. On the one hand, relatively large quantities of highly toxic chemicals are frequently used, which in themselves already represent a considerable risk for man and the environment, and on the other hand, epoxidation processes are often very highly exothermic, creating an increased explosion hazard when these reactions are carried out on the industrial scale. Obtaining official approval, under the terms of BimschG (German Air-borne Pollution Act), to operate industrial epoxidation plants therefore involves considerable expenditure.

The object of the present invention is therefore to provide a process for the epoxidation of olefins which avoids the abovementioned disadvantages. In particular, it should be possible to carry out this process in a simple, reproducible manner with increased safety for man and the environment and with good yields, and the reaction conditions should be very controllable.

Surprisingly, this object is achieved by the process according to the invention for the epoxidation of olefins, wherein the olefin, in liquid or dissolved form, is mixed with at least one oxidizing agent, in liquid or dissolved form, in at least one microreactor, the mixture is reacted for a certain residence time and the epoxide formed is optionally isolated from the reaction mixture.

Advantageous embodiments of the process according to the invention are described in the subclaims.

According to the invention, individual olefins or mixtures of at least two olefins can be reacted by the process claimed, although it is preferred to use only one olefin in the process according to the invention.

In terms of the invention, a microreactor is a reactor with a volume of ≦1000 μl in which the liquids and/or solutions are intimately mixed at least once. The volume of the microreactor is preferably ≦100 μl and particularly preferably ≦50 μl.

The microreactor is preferably made of thin interconnected silicon structures.

The microreactor is preferably a miniaturized continuous reactor and particularly preferably a static micromixer. Very particularly preferably, the microreactor is a static micromixer such as that described in the patent application with international publication number WO 96/30113, which is incorporated here by way of reference and constitutes part of the disclosure. Such a microreactor has small channels in which liquids and/or solutions of chemical compounds are mixed together by the kinetic energy of the flowing liquids and/or solutions.

The channels of the microreactor have a diameter preferably of 10 to 1000 μm, particularly preferably of 20 to 800 μm and very particularly preferably of 30 to 400 μm.

The liquids and/or solutions are pumped into the microreactor so as to flow through it at a rate preferably of 0.01 μl/min to 100 ml/min and particularly preferably of 1 μl/min to 1 ml/min.

According to the invention, the microreactor is preferably capable of being kept at a constant temperature.

According to the invention, the microreactor is preferably connected via an outlet to at least one detention section, preferably a capillary and particularly preferably a capillary capable of being kept at a constant temperature. After they have been thoroughly mixed in the microreactor, the liquids and/or solutions are transferred to this detention section or capillary to prolong their residence time.

In terms of the invention, the residence time is the time between the thorough mixing of the ducts and the work-up of the resulting reaction solution for analysis or isolation of the desired product(s).

The necessary residence time in the process according to the invention depends on a variety of parameters, e.g. the temperature or the reactivity of the educts. Those skilled in the art will be able to adapt the residence time to these parameters and thereby optimize the course of the reaction.

The residence time of the reaction solution in the system used, consisting of at least one microreactor and optionally a detection section, can be adjusted by the choice of flow rate of the liquids and/or solutions used.

Another preferred procedure is to pass the reaction mixture through two or more microreactors connected in series. The result is that, even with an increased flow rate, the residence time is prolonged and the components used in the epoxidation reaction are reacted so as to optimize the product yield of the desired epoxide(s).

In another preferred embodiment, the reaction mixture is passed through two or more microreactors arranged in parallel in order to increase the throughput.

In another preferred embodiment of the process according to the invention, the number and arrangement of the channels in one or more microreactors are varied to prolong the residence time so that, here again, with an increased flow rate, the yield of the desired epoxide(s) is optimized.

Preferably, the residence time of the reaction solution in the microreactor or, if appropriate, in the microreactor and the detention section is ≦15 hours, preferably ≦3 hours and particularly preferably ≦1 hour.

The process according to the invention can be carried out over a very wide temperature range which is limited essentially by the temperature resistance of the materials used to construct the microreactor and, if appropriate, the detention section, as well as other components, e.g. connectors and seals, and by the physical properties of the solutions and/or liquids used. Preferably, the process according to the invention is carried out at a temperature of −100 to +250° C., preferably of −78 to +150° C. and particularly preferably of 0 to +40° C.

The process according to the invention can be carried out continuously or batchwise, preferably continuously.

For carrying out the process according to the invention for the epoxidation of olefins, it is necessary for the epoxidation reaction to be carried out as far as possible in a homogeneous liquid phase containing no solid particles or only very small solid particles, as otherwise the channels in the microreactors become clogged.

The course of the epoxidation reaction in the process according to the invention can be monitored by various analytical methods known to those skilled in the art, and optionally regulated. The course of the reaction is monitored preferably by chromatography and particularly preferably by high performance liquid chromatography, and optionally regulated. This markedly improves control of the reaction compared with known processes.

After the reaction, the epoxides formed are optionally isolated. The epoxide(s) is (are) preferably isolated from the reaction mixture by extraction.

Any of the olefins known to those skilled in the art as epoxidation substrates can be used as olefins in the process according to the invention. The olefins are preferably selected from aliphatic, aromatic and heteroaromatic olefins, it being particularly preferred to use 1-phenylcyclohexene, cyclohexene or styrene.

Any of the aliphatic olefins known to those skilled in the art as suitable epoxidation substrates can be used as aliphatic olefins. These include linear, branched and cyclic olefins.

Any of the aromatic olefins known to those skilled in the art as suitable epoxidation substrates can be used as aromatic olefins. In terms of the invention, these include compounds and/or derivatives which have a monocyclic and/or polycyclic homoaromatic parent structure or a corresponding partial structure, e.g. in the form of substituents.

Any of the heteroaromatic olefins known to those skilled in the art as suitable epoxidation substrates and containing at least one heteroatom can be used as heteroaromatic olefins. In terms of the invention, heteroaromatic olefins include heteroaromatic compounds and/or derivatives thereof which have at least one monocyclic and/or polycyclic heteroaromatic parent structure or a corresponding partial structure, e.g. in the form of substituents. Heteroaromatic parent structures or partial structures particularly preferably comprise at least one oxygen, nitrogen and/or sulfur atom.

Any of the oxidizing agents known to those skilled in the art as suitable for epoxidations, or a mixture of at least two of these oxidizing agents, can be used as oxidizing agents in the process according to the invention. It is preferred to use only one oxidizing agent.

In another preferred embodiment of the invention, the oxidizing agent is at least one compound selected from inorganic and organic peroxides, hydrogen peroxide, chromyl compounds, chromium oxides, alkali metal hypochlorites, alkaline earth metal hypochlorites, N-bromosuccinimide, transition metal peroxo complexes, mixtures of peroxo compounds with organic acids and/or inorganic acids and/or Lewis acids, organic per-acids, inorganic per-acids and dioxirans, or a mixture of at least two of these oxidizing agents.

The inorganic peroxide used is preferably an ammonium peroxide, an alkali metal peroxide, particularly preferably sodium peroxide, an ammonium persulfate, an alkali metal persulfate, an ammonium perborate, an alkali metal perborate, an ammonium percarbonate, an alkali metal percarbonate, an alkaline earth metal peroxide, zinc peroxide or a mixture of at least two of these compounds.

The transition metal peroxo complex used is preferably a peroxo complex of iron, manganese, vanadium or molybdenum or a mixture of at least two of these peroxo complexes. A peroxo complex may also contain two or more identical or different metals, preferably selected from iron, manganese, vanadium and molybdenum.

Preferably, potassium peroxodisulfate with sulfuric acid is used as the peroxo compound with an inorganic acid, and hydrogen peroxide with boron trifluoride is used as the peroxo compound with a Lewis acid.

The organic per-acid used is preferably peroxybenzoic acid, m-chloroperoxybenzoic acid, p-nitroperoxybenzoic acid, magnesium monoperoxyphthalic acid, peroxyacetic acid, peroxymaleic acid, peroxytrifluoroacetic acid, peroxyphthalic acid, peroxylauric acid or a mixture of at least two of these per-acids.

Preferred dioxirans are dimethyldioxiran, methyl(trifluoromethyl)dioxiran and mixtures of these dioxirans.

The organic peroxide used is preferably tert-butyl hydroperoxide, cumene hydroperoxide, menthyl hydroperoxide, 1-methylcyclohexane hydroperoxide or a mixture of at least two of these organic peroxides.

The olefin can also be oxidized with optically active oxidizing agents or in the presence of optically active compounds to give optically active epoxides. The olefin is preferably oxidized with tert-butyl hydroperoxide in the presence of chiral reagents, preferably titanium tetraisopropoxide, diethyl (R,R)-tartrate and/or diethyl (S,S)-tartrate, to give optically active epoxides. It is also preferred to oxidize the olefin with the optically active (R,R)-trans-1,2-bis[(2-hydroxy-3,5-ditert-butylbenzylidene)amino]-cyclohexanemanganese dichloride or (S,S)-trans1,2-bis[(2-hydroxy-3,5-ditert-butylbenzylidene)amino]-cyclohexanemanganese dichloride (Jacobsen's catalyst) and dimethyldioxiran and/or sodium hypochlorite.

In the process according to the invention, the molar ratio of olefin to oxidizing agent used depends on the reactivity of the olefins used and of the oxidizing agents. The oxidizing agent and the olefin are preferably used in an equimolar ratio. In another preferred embodiment, the oxidizing agent is used in a 2-fold to 20-fold molar excess, particularly preferably in a 3-fold to 15-fold excess and very particularly preferably in a 4-fold to 10-fold excess, based on the olefin.

The selectivity of the reaction itself depends not only on the concentration of the reagents used but also on a number of other parameters, e.g. the temperature, the type of olefin used or the residence time. Those skilled in the art will be able to adapt the various parameters to the particular epoxidation to give the desired epoxide(s).

It is essential for the process according to the invention that the olefins and oxidizing agents used are either themselves liquid or present in dissolved form. If they are not already themselves in liquid form, they therefore have to be dissolved in a suitable solvent before the process according to the invention is carried out. The solvents used are preferably halogenated solvents, particularly preferably dichloromethane, chloroform, 1,2-dichloroethane or 1,1,2,2-tetrachloroethane, linear, branched or cyclic paraffins, particularly preferably pentane, hexane, heptane, octane, cyclopentane, cycloheptane or cyclooctane, linear, branched or cyclic ethers, particularly preferably diethyl ether, methyl tert-butyl ether, tetrahydrofuran or dioxane, aromatic solvents, particularly preferably toluene, xylenes, ligroin or phenyl ether, N-containing heterocyclic solvents, particularly preferably pyridine or N-methylpyrrolidone, or mixtures of at least two of the abovementioned solvents.

In the process according to the invention, the danger for man and the environment due to escaping chemicals is substantially reduced, thereby improving safety when handling hazardous substances. The epoxidation of olefins by the process according to the invention further affords better control of the reaction conditions, e.g. reaction time and reaction temperature, than is possible in the conventional processes. Also, in the process according to the invention, the explosion hazard associated with very highly exothermic epoxidations is markedly reduced. The temperature can be individually selected and kept constant in every volume element of the system. The course of the epoxidation reaction in the process according to the invention can be regulated very rapidly and precisely, making it possible to obtain the epoxides in very good and reproducible yields.

It is also particularly advantageous that the process according to the invention can be carried out continuously. This makes it more rapid and more cost-effective than conventional processes and any quantity of epoxides can be prepared without great expenditure on measurement and regulation.

The invention is illustrated below by means of an Example. This Example serves solely to illustrate the invention and does not limit the general inventive idea.

### EXAMPLE

### Epoxidation of 1-phenylcyclohexene to 1,2-epoxy-1-phenylcyclohexane

Phenylcyclohexene was epoxidized with m-chloroperbenzoic acid in a static micromixer (Technische Universität Ilmenau, Fakultät Maschinenbau, Dr.-Ing. Norbert Schwesinger, Postfach 100565, D-98684, Ilmenau) with external dimensions of 40 mm×25 mm×1 mm, which had a total of 11 mixing stages each with a volume of 0.125 μl. The total pressure loss was approx. 1000 Pa.

The static micromixer was connected via an outlet and an Omnifit medium pressure HPLC connector (Omnifit, Great Britain) to a Teflon capillary with an internal diameter of 0.49 mm and a length of 0.5 m. The reaction was carried out at 30° C., the static micromixer and the Teflon capillary being kept at this temperature in a thermostated jacketed vessel.

A 2 ml disposable injection syringe was filled with part of a solution of 150 mg (1 mmol) of 1-phenylcyclohexene in 5 ml of dichloromethane and another 2 ml syringe was filled with part of a solution of 2.15 g (12.5 mmol) of m-chloroperbenzoic acid in 25 ml of dichloromethane. The contents of both syringes were then transferred to the static micromixer by means of a metering pump (Harvard Apparatus Inc., Pump 22, South Natick, Mass., USA).

Before the reaction was carried out, the experimental set-up was calibrated in respect of the dependence of the residence time on the pump throughput. The residence time was adjusted to 4, 2 and 1 minute. The reactions were monitored by means of a Merck Hitachi LaChrom HPLC instrument and a Hewlett Packard GC-MS system. A quantitative conversion to the epoxidized product, 1,2-epoxy-1-phenylcyclohexane, was found for each of the three different residence times.

1. Process for the epoxidation of olefins, characterized in that at least one olefin, in liquid or dissolved form, is mixed with at least one oxidizing agent, in liquid or dissolved form, in at least one microreactor, the mixture is reacted for a certain residence time and the epoxide formed is optionally isolated from the reaction mixture.

2. Process according to claim 1, characterized in that the microreactor is a miniaturized continuous reactor.

3. Process according to claim 1 or 2, characterized in that the microreactor is a static micromixer.

4. Process according to one of claims 1 to 3, characterized in that the microreactor is connected via an outlet to a capillary, preferably a capillary capable of being kept at a constant temperature.

5. Process according to one of claims 1 to 4, characterized in that the volume of the microreactor is ≦100 μl, preferably ≦50 μl.

6. Process according to one of claims 1 to 5, characterized in that the microreactor is capable of being kept at a constant temperature.

7. Process according to one of claims 1 to 6, characterized in that the microreactor has channels with a diameter of 10 to 1000 μm, preferably of 20 to 800 μm and particularly preferably of 30 to 400 μm.

8. Process according to one of claims 1 to 7, characterized in that the reaction mixture flows through the microreactor at a rate of 0.01 μl/min to 100 ml/min, preferably of 1 μl/min to 1 ml/min.

9. Process according to one of claims 1 to 8, characterized in that the residence time of the compounds used in the microreactor or, if appropriate, in the microreactor and the capillary is ≦15 hours, preferably ≦3 hours and particularly preferably ≦1 hour.

10. Process according to one of claims 1 to 9, characterized in that it is carried out at a temperature of −100 to +250° C., preferably of −78 to +15020 C. and particularly preferably of 0 to +40° C.

11. Process according to one of claims 1 to 10, characterized in that the course of the reaction is monitored by chromatography, preferably by high performance liquid chromatography, and optionally regulated.

12. Process according to one of claims 1 to 11, characterized in that the epoxide formed is isolated from the reaction mixture by extraction or precipitation.

13. Process according to one of claims 1 to 12, characterized in that the oxidizing agent used is at least one oxidizing agent selected from inorganic and organic peroxides, hydrogen peroxide, chromyl compounds, chromium oxides, alkali metal hypochlorites, alkaline earth metal hypochlorites, N-bromosuccinimide, transition metal peroxo complexes, mixtures of peroxo compounds with organic acids and/or inorganic acids and/or Lewis acids, organic per-acids, inorganic peracids and dioxirans, or a mixture of at least two of these oxidizing agents.

14. Process according to claim 13, characterized in that the inorganic peroxide used is an ammonium peroxide, an alkali metal peroxide, preferably sodium peroxide, an ammonium persulfate, an alkali metal persulfate, an ammonium perborate, an alkali metal perborate, an ammonium percarbonate, an alkali metal percarbonate, an alkaline earth metal peroxide, zinc peroxide or a mixture of at least two of these compounds.

15. Process according to claim 13 or 14, characterized in that the transition metal peroxo complex used is a peroxo complex of iron, manganese, vanadium or molybdenum or a mixture of at least two of these peroxo complexes.

16. Process according to one of claims 13 to 15, characterized in that potassium peroxodisulfate with sulfuric acid is used as the peroxo compound with an inorganic acid, and hydrogen peroxide with boron trifluoride is used as the peroxo compound with a Lewis acid.

17. Process according to one of claims 13 to 16, characterized in that the organic per-acid used is peroxybenzoic acid, m-chloroperoxybenzoic acid, p-nitroperoxybenzoic acid, magnesium monoperoxyphthalic acid, peroxyacetic acid, peroxymaleic acid, peroxytrifluoroacetic acid, peroxyphthalic acid, peroxylauric acid or a mixture of at least two of these per-acids.

18. Process according to one of claims 13 to 17, characterized in that the dioxiran used is dimethyldioxiran, methyl(trifluoromethyl)dioxiran or a mixture of these dioxirans.

19. Process according to one of claims 13 to 18, characterized in that the organic peroxide used is tert-butyl hydroperoxide, cumene hydroperoxide, menthyl hydroperoxide, 1-methylcyclohexane hydroperoxide or a mixture of at least two of these compounds.

20. Process according to claim 19, characterized in that tert-butyl hydroperoxide is used in the presence of chiral reagents, preferably titanium tetraisopropoxide, diethyl (R,R)-tartrate or diethyl (S,S)-tartrate.

21. Process according to one of claims 1 to 20, characterized in that the olefin used is an aliphatic, cycloaliphatic, aromatic or heteroaromatic olefin, preferably 1-phenylcyclohexene, cyclohexene and/or styrene.

22. Process according to one of claims 1 to 21, characterized in that the molar ratio of olefin to oxidizing agent is equimolar or the oxidizing agent is used in a 2-fold to 20-fold molar excess, preferably in a 3-fold to 15-fold excess and particularly preferably in a 4-fold to 10-fold excess, based on the olefin.
